(19) United States
(12) Patent Application Publication
(10) Pub. No.: US 2002/0049249 A1
(43) Pub. Date: Apr. 25, 2002

(54) PPAR receptor activator compounds for treating cutaneous disorders/afflictions

(76) Inventors: Jean-Michel Bernardon, Le Rouret (FR); Serge Michel, Roquefort-Les-Pins (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/933,818

(22) Filed: Aug. 22, 2001

Related U.S. Application Data
(63) Continuation of application No. PCT/FR00/03646, filed on Dec. 21, 2000.

(30) Foreign Application Priority Data
Dec. 22, 1999 (FR) 99/16270

(51) Int. Cl. A61K 31/385; A61K 31/39; A61K 31/357; A61K 31/352; A61K 31/195; A61K 31/192

(52) U.S. Cl. 514/434; 514/452; 514/456; 514/567; 514/569

(57) ABSTRACT

Compounds of formula (I):

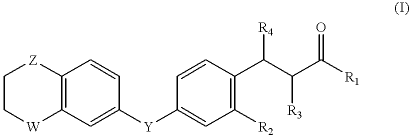

characteristically activators of receptors of PPAR type, are well suited for treating cutaneous disorders/afflictions, notably disorders of the barrier function of the skin, more particularly disorders of the secretion of epidermal lipids, photodermatoses or ulcers, and/or disorders of the metabolism of lipids.

### CROSS-REFERENCE TO PRIORITY/PCT APPLICATIONS

This application claims priority under 35 U.S.C. § 119 of FR-99/16270, filed Dec. 22, 1999, and is a continuation of PCT/FR00/03646, filed Dec. 21, 2000 and designating the United States (published in the French language on Jun. 28, 2001 as WO 01/45664 A2; the title and abstract were also published in English), both hereby expressly incorporated by reference.

### CROSS-REFERENCE TO COMPANION APPLICATION

Copending application Ser. No. ______ [Attorney Docket No. 016800-452], filed concurrently herewith and assigned to the assignee hereof.

### BACKGROUND OF THE INVENTION

Technical Field of the Invention

The present invention relates to the administration of polycyclic aromatic compounds, or cosmetic/pharmaceutical compositions comprised thereof, for the treatment of cutaneous disorders/afflictions, such as disorders of the barrier function, more particularly disorders of the secretion of epidermal lipids, photodermatoses or ulcers, and/or disorders of the metabolism of lipids.

The present invention also relates to a cosmetic/pharmaceutical regime or regimen for restoring the barrier function of the skin and more particularly for regulating the metabolism of cutaneous lipids, comprising topically applying at least one compound of formula (I) below, more particularly as activator of receptors of PPAR type, onto the skin.

### SUMMARY OF THE INVENTION

It has now unexpectedly and surprisingly been determined that certain polycyclic aromatic compounds, notably those described in EP-722,928, exhibit an antiproliferative effect and elicit marked activity with regard to the transactivation of receptors of PPAR type.

The present invention thus features administration of an effective amount of at least one polycyclic aromatic compound, more particularly as activators of receptors of PPAR type, or compositions comprised thereof, for the treatment of cutaneous disorders/afflictions such as disorders of the barrier function of the skin, more particularly disorders of the secretion of epidermal lipids, photodermatoses or ulcers, and/or disorders of the metabolism of lipids.

This invention also features a cosmetic regime/regimen for restoring the barrier function of the skin and more particularly for regulating the metabolism of cutaneous lipids, comprising topically applying at least one compound of formula (I), more particularly as activator of receptors of PPAR type, onto the skin.

The subject compounds have the structural formula (I):

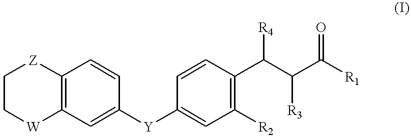

in which R₁ is a hydrogen atom or an —OR₅ radical, wherein R₅ is as defined below; R₂ is a hydrogen atom or a lower alkyl radical; R₃ and R₄, which may be identical or different, are each a hydrogen atom or a lower alkyl radical, with the proviso that R₂ and R₃, may together form, with the carbon atoms from which they depend, a naphthalene ring with the adjacent benzene ring; Y is an oxygen atom, an S(O)ₙ radical or an N—R₆ radical, wherein n and R₆ are as defined below; Z and W, which may be identical or different, are each —CR₇R₈—, —O— or —S(O)ₘ, wherein m R₇ and R₈ are as defined below; R₅ is a hydrogen atom, a linear or branched alkyl radical having from 1 to 20 carbon atoms, or a mono- or polyhydroxyalkyl radical; R₆ is a hydrogen atom or a lower alkyl radical; R₇ and R₈, which may be identical or different, are each a hydrogen atom or a lower alkyl radical; n is 0, 1 or 2; m is 0, 1 or 2; and include the salts and chiral analogs thereof. Such salts include those of an alkali or alkaline earth metal, or of zinc, or of an organic amine.

### DETAILED DESCRIPTION OF BEST MODE AND SPECIFIC/PREFERRED EMBODIMENTS OF THE INVENTION

More particularly according to the present invention, by the term “lower alkyl radical” is intended a radical having 1 to 6 carbon atoms, preferably the methyl, ethyl, isopropyl, butyl, tert-butyl and hexyl radicals.

And exemplary linear or branched alkyl radicals having from 1 to 20 carbon atoms are the methyl, ethyl, propyl, 2-ethylhexyl, octyl, docecyl, hexadecyl and octadecyl radicals.

By the term “monohydroxyalkyl radical” is intended a radical having 1 to 6 carbon atoms and preferably having from 2 to 3 carbon atoms, in particular a 2-hydroxyethyl, 2-hydroxypropyl or 3-hydroxypropyl radical.

By the term “polyhydroxyalkyl radical” is intended a radical having from 3 to 6 carbon atoms and 2 to 5 hydroxyl groups, such as the 2,3-dihydroxypropyl, 2,3,4-trihydroxybutyl and 2,3,4,5-tetrahydroxypentyl radicals and the pentaerythritol residue.

Particularly exemplary compounds of formula (I) are the following:

Compound 1: 6-(5,5,8,8-tetramethyl-5,6,7,8-tetrahydronaphth-2-yloxy)naphthalene-2-carboxylic acid;

Compound 2: 3-[4-(5,5,8,8-tetramethyl-5,6,7,8-tetrahydronaphth-2-yloxy)phenyl]acrylic acid;

Compound 3: 6-(5,5,8,8-tetramethyl-5,6,7,8-tetrahydronaphth-2-ylsulfanyl)naphthalene-2-carboxylic acid;

Compound 4: 3-[4-(5,5,8,8-tetramethyl-5,6,7,8-tetrahydronaphth-2-yloxy)phenyl]but-2-enoic acid;

Compound 5: 6-(5,5,8,8-tetramethyl-5,6,7,8-tetrahydronaphth-2-ylamino)naphthalene-2-carboxylic acid.

According to the present invention, the compound of formula (I) which is more particularly preferred is Compound 3: 6-(5,5,8,8-tetramethyl-5,6,7,8-tetrahydronaphth-2-ylsulfanyl)naphthalene-2-carboxylic acid.

The compounds of formula (I) can be prepared, in particular, by the methodology described in EP-722,928.

The compounds of the invention exhibit activating properties with regard to receptors of PPAR type. The receptors of PPAR type are receptors which belong to the family of steroid nuclear receptors.

By the term “activator of receptors of PPAR type” is intended any compound that exhibits in a transactivation test, such as described in Kliewer et al., Nature, 358, 771-774 (1992), an AC₅₀ of less than or equal to 10 μM. The activator of receptors of PPAR type preferably exhibits an AC₅₀ of less than or equal to 2 μM and advantageously of less than or equal to 1 μM.

An AC₅₀ is the concentration of “activator” compound necessary to exhibit 50% of the activity of a reference molecule. This activity is determined employing an enzyme (luciferase) which is a reporter of the activation due to the compound via one of the PPAR receptors.

The activity of receptors of PPAR type is the subject of numerous studies and publications. Exemplary is the publication entitled “Differential Expression of Peroxisome Proliferator-Activated Receptor Subtypes During the Differentiation of Human Keratinocytes,” Michel Rivier et al., J. Invest. Dermatol., 111, p. 1116-1121 (1998), in which a large number of bibliographic references relating to receptors of PPAR type are listed.

The use of activators of receptors of PPAR-α type for restoring the barrier function and more particularly disorders of the secretion of epidermal lipids, promoting epidermal differentiation and inhibiting epidermal proliferation, is described in WO 98/32444.

Furthermore, the administration of activators of receptors of PPAR-αand/or PPAR-y type for treating cutaneous disorders related to an anomaly in the differentiation of epidermal cells has been described by Michel Rivier et al., J. Invest. Dermatol., 111, p. 1116-1121 (1998).

It has also been described in WO 96/33724, that compounds which are selective for PPAR-γ receptors, such as a prostaglandin-J2 or -D2, are potential active principles for the treatment of obesity and diabetes.

Pharmaceutical compositions comprising at least one compound of formula (I) are thus well suited for the treatment of cutaneous disorders/afflictions, such as disorders of the skin barrier function, more particularly disorders of the secretion of epidermal lipids, photodermatoses or ulcers, and/or disorders of the metabolism of lipids.

Particulary exemplary disorders of the barrier function of the skin are, more especially, disorders of the secretion of epidermal lipids, of skin disorders in premature babies born before 33 weeks, chapped lips or blisters resulting from mechanical friction.

Exemplary ulcers are ulcers and erosions due to chemical or thermal burns, bullous disorders or vascular or ischaemia disorders, including venous, arterial, embolic or diabetic ulcers.

And exemplary conditions of the metabolism of lipids are obesity, hyperlipidaemia or non-insulin-dependent diabetes.

The compositions according to the invention can be administered via the enteral, parenteral or topical or ocular route, for such period of time as required to elicit the desired response. The pharmaceutical compositions are preferably packaged in a form suitable for application by the topical route.

The subject compositions, more particularly the pharmaceutical compositions, can be provided, for the enteral route, in the form of tablets, including sugar-coated tablets, hard gelatin capsules, syrups, suspensions, solutions, powders, granules, emulsions or lipid or polymeric microspheres or nanospheres or vesicles which permit controlled release. The subject compositions can be provided, for the parenteral route, in the form of solutions or suspensions for infusion or for injection.

The subject compounds according to the invention are generally administered at a daily dose of approximately 0.001 mg/kg to 100 mg/kg of body weight, taken on 1 to 3 occasions.

The pharmaceutical compositions according to the invention, for the topical route, are more preferably for the treatment of the skin and mucous membranes and can be provided in the form of salves, creams, emulsions, milks, ointments, powders, impregnated pads, solutions, gels, sprays, lotions or suspensions. Same can also be provided in the form of lipid or polymeric microspheres or nanospheres or vesicles or of polymeric patches and hydrogels which permit controlled release. The compositions for topical application can be provided either in anhydrous form or in aqueous form.

The subject compounds are administered via the topical route at a concentration generally ranging from 0.001% to 10% by weight, preferably from 0.01 to 1% by weight, with respect to the total weight of the composition.

The compounds of formula (I) according to the invention also find application in the cosmetics field, in particular in body and hair hygiene and more particularly for restoring the barrier function of the skin and more particularly for regulating and/or restoring the metabolism of cutaneous lipids. In comparison with the products known previously, these compounds of formula (I) present the advantage of additionally exhibiting other advantageous properties, in particular anti-inflammatory or soothing properties, which makes them less irritating and therefore better tolerated compounds.

The cosmetic compositions according to the invention, comprising, in a cosmetically acceptable vehicle, diluent or carrier, at least one compound of formula (I), one of its optical or geometrical isomers or one of its salts, can be provided, in particular, in the form of a cream, milk, lotion, emulsion, gel, lipid or polymeric microspheres or nanospheres or vesicles, soap or shampoo.

The concentration of compound of formula (I) in the cosmetic compositions advantageously ranges from 0.001% to 3% by weight.

The compositions according to the present invention can, of course, additionally comprise inert or even pharmacodynamically active additives or adjuvants, or combinations of these additives and adjuvants, and in particular: wetting agents; depigmenting agents, such as hydroquinone, azelaic acid, caffeic acid or kojic acid; emollients; moisturizing agents, such as glycerol, PEG 400, thiamorpholinone and derivatives thereof, or urea; antiseborrhoeic or antiacne agents, such as S-carboxymethylcysteine, S-benzylcysteamine, their salts or derivatives thereof, or benzoyl peroxide; antifungal agents, such as ketoconazole or 4,5-polymethylene-3-isothiazolidones; antibacterials; carotenoids and in particular β-carotene; antipsoriatic agents, such as anthralin and derivatives thereof; eicosa-5,8,11,14-tetraynoic and eicosa-5,8,11-triynoic acids, their esters and amides, and also the retinoids. The subject compounds of formula (I) can also be formulated with vitamins D or derivatives thereof, with corticosteroids, with agents for combating free radicals, with α-hydroxy or α-keto acids or derivatives thereof, or with ion channel blockers.

These compositions can also comprise flavor-improving agents, preservatives, such as esters of para-hydroxybenzoic acid, stabilizing agents, moisture-regulating agents, pH-regulating agents, agents for modifying osmotic pressure, emulsifying agents, UV-A and UV-B screening agents, or antioxidants, such as α-tocopherol, butylhydroxyanisole or butylhydroxy-toluene.

One skilled in this art will of course take care to select the optional compound or compounds to be added to these compositions such that the advantageous properties intrinsically associated with the present invention are not, or not substantially, detrimentally affected by the envisaged addition.

In order to further illustrate the present invention and the advantages thereof, the following specific examples are given, it being understood that same are intended only as illustrative and in nowise limitative.

In said examples to follow, all parts and percentages are given by weight, unless otherwise indicated.

### EXAMPLE 1

Various results of biological tests which illustrate the properties of transactivation of PPAR receptors of the compounds of the invention are reported in these examples.

The comparative examples correspond to compounds which are disclosed in EP-722,928 but which do not verify the conditions of the compounds of formula (I).

The biological tests carried out correspond to those described above. The method used to determine the AC₅₀ values was that described in Kliewer et al., Nature, 358, 771-774 (1992). Thus, the activating power via PPAR-α, PPAR-γ or PPAR-δ of molecules can be evaluated with a transactivation test in which HeLa cells were cotransfected with an expression vector encoding these receptors and a reporter plasmid comprising a PPRE response element cloned upstream of a portion of a promoter of the SV40 virus and of the luciferase gene. The cotransfected cells were treated for 24 hours with the molecules to be tested and the activity of the luciferase was determined by luminescence.

Reference 1, the reference molecule for PPAR-α receptors, was [4-chloro-6-(2,3-dimethyl-phenylamino) pyrimidin-2-ylsulfanyl]acetic acid; Reference 2, the reference molecule for PPAR-δ and PPAR-Γ receptors, was 5-{4-[2-(methylpyrid-2-ylamino)ethoxy]benzyl}thiazolidine-2,4-dione.

Comparative Example 1 was 2-methyl-4-[4-(5,5,8,8,-tetramethyl-5,6,7,8-tetrahydronaphth-2-yloxy)benzylidine]-4H-oxazol-5-one. Comparative Example 2 was 2-acetylamino-3-[4-(5,5,8,8-tetramethyl-5,6,7,8-tetrahydronaphth-2-yloxy)phenyl]-acrylic acid.

The results obtained in the tests of transactivation of receptors of PPAR type are combined in the following table:

| Compounds | α | γ | β |
|---|---|---|---|
| Reference 1 | 100* (1.4)** | n.a | n.a |
| Reference 2 | n.a | 100(0.07) | 100(0.13) |
| Compound 1 | 18 | 23 | 152(0.7) |
| Compound 2 | 12 | 18 | 204(0.9) |
| Compound 3 | 24 | 40 | 172(0.2) |
| Compound 4 | 12 | 0 | 56 |
| Compound 5 | 25 | 69 | 328(7) |
| Comparative | 5 | 0 | 7 |
| Example 1 | | | |
| Comparative | 7 | 4 | 0 |
| Example 2 | | | |

n.a connotes “non active”
*% of activation
0** AC₅₀ in μM

These results evidence the activation of the compounds of the invention for the various subtypes of receptors of PPAR type: PPAR-α, PPAR-β and PPAR-γ.

### EXAMPLE 2

Various specific compositions based on the compounds according to the invention were formulated:

A—Oral Route:

(a) 0.2 g tablet:

| | |
|---|---|
| Compound 1 | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

(b) Oral Suspension in 5 ml Vials:

(b) Oral suspension in 5 ml vials:

| | | |
|---|---|---|
| Compound 5 | | 0.001 g |
| Glycerol | | 0.500 g |
| 70% Sorbitol | | 0.500 g |
| Sodium saccharin | | 0.010 g |
| Methyl para-hydroxybenzoate | | 0.040 g |
| Flavoring | q.s. | |
| Purified water | q.s. for | 0.001 g |

(c) 0.8 g Tablet:

(c) 0.8 g tablet:

| | |
|---|---|
| Compound 2 | 0.500 g |
| Pregelatinized starch | 0.100 g |
| Microcrystalline cellulose | 0.115 g |
| Lactose | 0.075 g |
| Magnesium stearate | 0.010 g |

(d) Oral Suspension in 10 ml Vials:

(d) Oral suspension in 10 ml vials:

| | | |
|---|---|---|
| Compound 4 | | 0.200 g |
| Glycerol | | 1.000 g |
| 70% Sorbitol | | 1.000 g |
| Sodium saccharin | | 0.010 g |
| Methyl para-hydroxybenzoate | | 0.080 g |
| Flavoring | q.s. | |
| Purified water | q.s. for | 10 ml |

B—Topical Route:

(a) Salve:

(a) Salve:

| | |
|---|---|
| Compound 1 | 0.020 g |
| Isopropyl myristate | 81.700 g |
| Fluid liquid petrolatum | 9.100 g |
| Silica (“Aerosil 200” marketed by Degussa) | 0.020 g |

(b) Salve:

(b) Salve:

| | | |
|---|---|---|
| Compound 2 | | 0.300 g |
| White petrolatum, pharmaceutical | | |
| grade | q.s. for | 100 g |

(c) Nonionic Water-in-oil Cream:

(c) Nonionic water-in-oil cream:

| | | |
|---|---|---|
| Compound 1 | | 0.100 g |
| Mixture of emulsified lanolin | | |
| alcohols, of waxes and of oils | | |
| (“Anhydrous Eucerin”, marketed by BDF) | | 39.900 g |
| Methyl para-hydroxybenzoate | | 0.075 g |
| Propyl para-hydroxybenzoate | | 0.075 g |
| Sterile demineralized water | q s. for | 100 g |

(d) Lotion:

(d) Lotion:

| | |
|---|---|
| Compound 3 | 0.100 g |
| Polyethylene glycol (PEG 400) | 69.900 g |
| 95% Ethanol | 30.000 g |

(e) Hydrophobic Salve:

(e) Hydrophobic salve:

| | | |
|---|---|---|
| Compound 5 | | 0.300 g |
| Isopropyl myristate | | 36.400 g |
| Silicone oil (“Rhodorsil 47 V 300”, | | 36.400 g |
| marketed by Rhone-Poulenc) | | |
| Beeswax | | 13.600 g |
| Silicone oil (“Abil 300.000 cst” marketed | q.s. for | 100 g |
| by Goldschmidt) | | |

(f) Nonionic Oil-in-water Cream:

| | | | |
|---|---|---|---|
| Compound 2 | | 1.000 | g |
| Cetyl alcohol | | 4.000 | g |
| Glyceryl monostearate | | 2.500 | g |
| PEG 50 stearate | | 2.500 | g |
| Karite butter | | 9.200 | g |
| Propylene glycol | | 2.000 | g |
| Methyl para-hydroxybenzoate | | 0.075 | g |
| Propyl para-hydroxybenzoate | | 0.075 | g |
| Sterile demineralized water | q.s. for | 100 | g |

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for treating the cutaneous disorders/afflictions of an individual in need of such treatment, comprising administering to such individual, for such period of time as required to elicit the desired response, a thus-effective amount of at least one polycyclic aromatic compound having the structural formula (I) 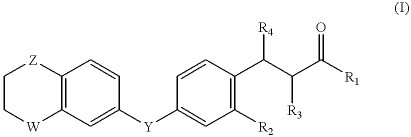
in which R₁ is a hydrogen atom or an —OR₅ radical, wherein R₅ is as defined below; R₂ is a hydrogen atom or a lower alkyl radical; R₃ and R₄, which may be identical or different, are each a hydrogen atom or a lower alkyl radical, with the proviso that R₂ and R₃, may together form a naphthalene ring with the adjacent benzene ring; Y is an oxygen atom, an S(O)ₙ radical or an N—R₆ radical, wherein n and R₆ are as defined below; Z and W, which may be identical or different, are each —CR₇R₈—, —O— or —S(O)ₘ, wherein m, R₇ and R₈ are as defined below; R₅ is a hydrogen atom, a linear or branched alkyl radical having from 1 to 20 carbon atoms, or a mono- or polyhydroxyalkyl radical; R₆ is a hydrogen atom or a lower alkyl radical; R₇ and R₈, which may be identical or different, are each a hydrogen atom or a lower alkyl radical; n is 0, 1 or 2; m is 0, 1 or 2; or salt or chiral analog thereof.

2. A regime or regimen for treating disorders of the barrier function of human skin, disorders of the secretion of epidermal lipids, photodermatoses or ulcers, and/or disorders of the metabolism of lipids, comprising administering to an individual in need of such treatment, for such period of time as required to elicit the desired response, a thus-effective amount of at least one polycyclic aromatic compound having the structural formula (I): 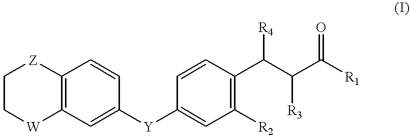
in which R₁ is a hydrogen atom or an —OR₅ radical, wherein R₅ is as defined below; R₂ is a hydrogen atom or a lower alkyl radical; R₃ and R₄, which may be identical or different, are each a hydrogen atom or a lower alkyl radical, with the proviso that R₂ and R₃, may together form a naphthalene ring with the adjacent benzene ring; Y is an oxygen atom, an S(O)ₙ radical or an N—R₆ radical, wherein n and R₆ are as defined below; Z and W, which may be identical or different, are each —CR₇R₈—, —O— or —S(O)ₘ, wherein m, R₇ and R₈ are as defined below; R₅ is a hydrogen atom, a linear or branched alkyl radical having from 1 to 20 carbon atoms, or a mono- or polyhydroxyalkyl radical; R₆ is a hydrogen atom or a lower alkyl radical; R₇ and R₈, which may be identical or different, are each a hydrogen atom or a lower alkyl radical; n is 0, 1 or 2; m is 0, 1 or 2; or salt or chiral analog thereof.

3. The regime or regimen as defined by claim 2, comprising treating skin disorders in premature babies born before 33 weeks, chapped lips, or blisters.

4. The regime or regimen as defined by claim 2, comprising treating ulcers and erosions due to chemical or thermal burns, a bullous disorder, or a vascular or ischaemia disorder.

5. The regime or regimen as defined by claim 4, comprising treating venous, arterial, embolic or diabetic ulcers.

6. The regime or regimen as defined by claim 2, comprising treating obesity, hyperlipidaemia or non-insulin-dependent diabetes.

7. The regime or regimen as defined by claim 1, said at least one polycyclic aromatic compound (I) comprising a PPAR receptor activator.

8. The regime or regimen as defined by claim 2, said at least one polycyclic aromatic compound (I) comprising a PPAR receptor activator.

9. The regime or regimen as defined by claim 2, said at least one polycyclic aromatic compound (I) comprising at least one lower alkyl radical selected from the group consisting of methyl, ethyl, isopropyl, butyl, tert-butyl and hexyl radicals.

10. The regime or regimen as defined by claim 2, said at least one polycyclic aromatic compound (I) comprising at least one linear or branched alkyl radical having from 1 to 20 carbon atoms selected from the group consisting of methyl, ethyl, propyl, 2-ethylhexyl, octyl, dodecyl, hexadecyl and octadecyl radicals.

11. The regime or regimen as defined by claim 2, said at least one polycyclic aromatic compound (I) comprising at least one monohydroxyalkyl radical selected from the group consisting of 2-hydroxyethyl, 2-hydroxypropyl and 3-hydroxypropyl radicals.

12. The regime or regimen as defined by claim 2, said at least one polycyclic aromatic compound (I) comprising at least one polyhydroxyalkyl radical selected from the group consisting of 2,3-dihydroxypropyl, 2,3,4-tri-hydroxybutyl and 2,3,4,5-tetrahydroxypentyl radicals and the pentaerythritol residue.

13. The regime or regimen as defined by claim 2, said at least one polycyclic aromatic compound (I) comprising Compound 1: 6-(5,5,8,8-tetramethyl-5,6,7,8-tetrahydronaphth-2-yloxy)naphthalene-2-carboxylic acid; Compound 2: 3-[4-(5,5,8,8-tetramethyl-5,6,7,8-tetrahydronaphth-2-yloxy)phenyl]acrylic acid; Compound 3: 6-(5,5,8,8-tetramethyl-5,6,7,8-tetrahydronaphth-2-ylsulfanyl)naphthalene-2-carboxylic acid; Compound 4: 3-[4-(5,5,8,8-tetramethyl-5,6,7,8-tetrahydronaphth-2-yloxy)phenyl]but-2-enoic acid; or Compound 5: 6-(5,5,8,8-tetramethyl-5,6,7,8-tetrahydronaphth-2-ylamino)naphthalene-2-carboxylic acid.

14. The regime or regimen as defined by claim 2, said at least one polycyclic aromatic compound (I) comprising 6-(5,5,8,8-tetramethyl-5,6,7,8-tetrahydronaphth-2-ylsulfanyl)naphthalene-2-carboxylic acid.

15. The regime or regimen as defined by claim 1, comprising administering said at least one polycyclic aromatic compound (I) via enteral or parenteral route.

16. The regime or regimen as defined by claim 1, comprising administering said at least one polycyclic aromatic compound (I) via topical or ocular route.

17. The regime or regimen as defined by claim 1, comprising coadministering to such individual an effective amount of at least one retinoid, vitamin D or derivative thereof, corticosteroid, agent for combating free radicals, α-hydroxy or α-keto acid or derivative thereof, or ion channel blocker.

18. Tablets, capsules, syrup, suspension, solution, powder, granules, emulsion, lipid or polymeric microspheres, nanospheres or vesicles comprising an amount effective for treating disorders of the barrier function of human skin, disorders of the secretion of epidermal lipids, photodermatoses or ulcers, and/or disorders of the metabolism of lipids, of at least one polycyclic aromatic compound having the structural formula (I): 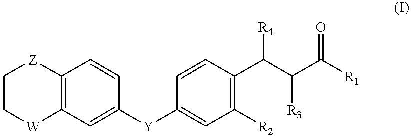
in which R₁ is a hydrogen atom or an —OR₅ radical, wherein R₅ is as defined below; R₂ is a hydrogen atom or a lower alkyl radical; R₃ and R₄, which may be identical or different, are each a hydrogen atom or a lower alkyl radical, with the proviso that R₂ and R₃, may together form a naphthalene ring with the adjacent benzene ring; Y is an oxygen atom, an S(O)ₙ radical or an N—R₆ radical, wherein n and R6 are as defined below; Z and W, which may be identical or different, are each —CR₇R₈—, —O— or —S(O)ₘ, wherein m, R₇ and R₈ are as defined below; R₅ is a hydrogen atom, a linear or branched alkyl radical having from 1 to 20 carbon atoms, or a mono- or polyhydroxyalkyl radical; R₆ is a hydrogen atom or a lower alkyl radical; R₇ and R₈, which may be identical or different, are each a hydrogen atom or a lower alkyl radical; n is 0, 1 or 2; m is 0, 1 or 2, or salt or chiral analog thereof, formulated into an enterally/parenterally administrable, cosmetically/pharmaceutically acceptable vehicle, diluent or carrier therefor.

19. A salve, cream, emulsion, milk, ointment, powder, impregnated pad, solution, gel, spray, lotion, suspension, lipid or polymeric microspheres, nanospheres, vesicles, patch, hydrogel, soap or shampoo comprising an amount effective for treating disorders of the barrier function of human skin, disorders of the secretion of epidermal lipids, photodermatoses or ulcers, and/or disorders of the metabolism of lipids, of at least one polycyclic aromatic compound having the structural formula (I): 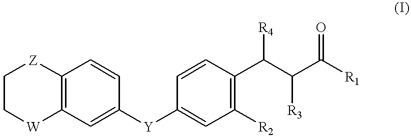
in which R₁ is a hydrogen atom or an —OR₅ radical, wherein R₅ is as defined below; R₂ is a hydrogen atom or a lower alkyl radical; R₃ and R₄, which may be identical or different, are each a hydrogen atom or a lower alkyl radical, with the proviso that R₂ and R₃, may together form a naphthalene ring with the adjacent benzene ring; Y is an oxygen atom, an S(O)ₙ radical or an N—R₆ radical, wherein n and R₆ are as defined below; Z and W, which may be identical or different, are each —CR₇R₈—, —O— or —S(O)ₘ, wherein m, R₇ and R₈ are as defined below; R₅ is a hydrogen atom, a linear or branched alkyl radical having from 1 to 20 carbon atoms, or a mono- or polyhydroxyalkyl radical; R₆ is a hydrogen atom or a lower alkyl radical; R₇ and R₈, which may be identical or different, are each a hydrogen atom or a lower alkyl radical; n is 0, 1 or 2; m is 0, 1 or 2, or salt or chiral analog thereof, formulated into a topically applicable, cosmetically/pharmaceutically acceptable vehicle, diluent or carrier therefor.

20. The formulation as defined by claim 18, further comprising an effective amount of at least one retinoid, vitamin D or derivative thereof, corticosteroid, agent for combating free radicals, α-hydroxy or α-keto acid or derivative thereof, or ion channel blocker.

21. The formulation as defined by claim 19, further comprising an effective amount of at least one retinoid, vitamin D or derivative thereof, corticosteroid, agent for combating free radicals, α-hydroxy or α-keto acid or derivative thereof, or ion channel blocker.
